(19) United States
(12) Patent Application Publication
(10) Pub. No.: US 2023/0397803 A1
(43) Pub. Date: Dec. 14, 2023

(54) DENTAL RETRACTOR

(71) Applicants: Brian Halleck, Santa Barbara, CA (US); Michael Shinn, Santa Barbara, CA (US); Jacob Swartz, Santa Barbara, CA (US); James Hirsch, Santa Barbara, CA (US); Amy Kerr, Hot Springs, SD (US); Shaun Valentine, Fort Thomas, KY (US)

(72) Inventors: Brian Halleck, Santa Barbara, CA (US); Michael Shinn, Santa Barbara, CA (US); Jacob Swartz, Santa Barbara, CA (US); James Hirsch, Santa Barbara, CA (US); Amy Kerr, Hot Springs, SD (US); Shaun Valentine, Fort Thomas, KY (US)

(21) Appl. No.: 17/839,414

(22) Filed: Jun. 13, 2022

(51) Int. Cl. A61B 1/24; A61C 5/90

(52) U.S. Cl. CPC A61B 1/24; A61C 5/90

(57) ABSTRACT

A dental retractor has molded flexural support members to provide upper lip, lower lip, and cheek retraction. The retraction provides unobstructed access and visibility for facial/labial areas of the mouth during dental procedures. The lips are prevented from touching gingival tissue and tooth structure. Lips and soft tissue are retracted and held safely away from dental tools. Labial retraction is provided by flexible members which, when positioned in the use position, provide elevation and retraction for the labial surfaces both maxillary and mandibular, the counteractive forces of the maxillary and mandibular support members keep the lips retracted and away from treatment areas. As the flexural support members are flexed for labial retraction, the medial portions of the support members bilaterally retract the cheeks due to the designed forces in the support members.

### BACKGROUND OF THE INVENTION

### 1. Field of the Invention

Embodiments of the invention relate generally to dental mouthpieces. More particularly, embodiments of the invention relate to a dental retractor providing upper lip, lower lip, and cheek retraction.

### 2. Description of Prior Art and Related Information

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

In performing dental procedures, there is often a need to perform multiple functions at the same time. Such functions can include keeping the patient's mouth open, retracting tissue such as a patient's lips and the like. Conventional lip and cheek retractors may be uncomfortable for user and may be difficult to use and deploy.

In view of the foregoing, there is a need for improved dental retractors that can provide proper retraction while being comfortable for the patient and easy to use for the provider.

### SUMMARY OF THE INVENTION

Embodiments of the present invention provide a dental retractor comprising an upper lip saddle; a lower lip saddle, each of the upper lip saddle and the lower lip saddle having an interior and an exterior, the interior being positioned between a user's lip and gum when the dental retractor is placed on a user, the exterior being positioned on an outer portion of the user's lips with the dental retractor is placed on the user; a first support member joining a left side of the interior of the lower lip saddle with a left side of the interior of the upper lip saddle; and a second support member joining a right side of the interior of the lower lip saddle with a right side of the interior of the upper lip saddle, wherein the upper lip saddle and the lower lip saddle being disposed along a longitudinal axis when the dental retractor is at rest and not acted upon by any force; the first and second support members provide resilient bending of the upper lip saddle toward the lower lip saddle; and the first and second support members are resiliently bend outward from a space between the upper lip saddle and the lower lip saddle when the dental retractor is bent to a use position.

Embodiments of the present invention further provide a dental retractor comprising an upper lip saddle; a lower lip saddle, each of the upper lip saddle and the lower lip saddle having an interior and an exterior, the interior being positioned between a user's lip and gum when the dental retractor is placed on a user, the exterior being positioned on an outer portion of the user's lips with the dental retractor is placed on the user; a tab on a central portion of each of the exterior of the upper lip saddle and the lower lip saddle; a first support member joining a left side of the interior of the lower lip saddle with a left side of the interior of the upper lip saddle; and a second support member joining a right side of the interior of the lower lip saddle with a right side of the interior of the upper lip saddle, wherein the upper lip saddle and the lower lip saddle being disposed along a longitudinal axis when the dental retractor is at rest and not acted upon by any force; the first and second support members provide resilient bending of the upper lip saddle toward the lower lip saddle; the first and second support members are resiliently bend outward from a space between the upper lip saddle and the lower lip saddle when the dental retractor is bent to a use position; and an upper half of the dental retractor, having the upper lip saddle, is symmetrical with a lower half of the dental retractor, having the lower lip saddle.

Embodiments of the present invention further provide a method of retracting a user's lips and cheeks comprising squeezing the tabs of the dental retractor as described herein toward each other to resiliently bend the dental retractor into an angle of about 90 degrees; and inserting the dental retractor into a user's mouth, where the upper lip saddle is positioned about a user's upper lip, the lower lip saddle is positioned about a user's lower lip, and each of the first and second support members press against an interior of respective left and right cheeks of a user.

These and other features, aspects and advantages of the present invention will become better understood with reference to the following drawings, description and claims.

### BRIEF DESCRIPTION OF THE DRAWINGS

Some embodiments of the present invention are illustrated as an example and are not limited by the figures of the accompanying drawings, in which like references may indicate similar elements.

FIG. 1 illustrates a perspective view of a dental retractor according to an exemplary embodiment of the present invention;

FIG. 2 illustrates a side view of the dental retractor of FIG. 1;

FIG. 3 illustrates an end view of the dental retractor of FIG. 1;

FIG. 4 illustrates a top view of the dental retractor of FIG. 1;

FIG. 5 illustrates a bottom view of the dental retractor of FIG. 1;

FIG. 6 illustrates movement of the dental retractor of FIG. 1 between a storage position to an insertion position;

FIG. 7 illustrates the dental retractor of FIG. 1 used on a patient; and

FIG. 8 illustrates the dental retractor of FIG. 1 used on a patient with an additional dental mouthpiece.

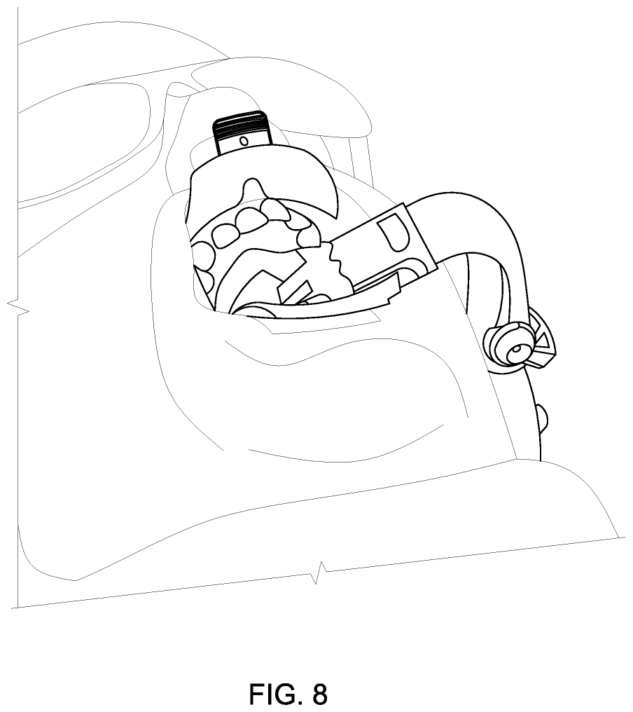

Unless otherwise indicated illustrations in the figures are not necessarily drawn to scale.

The invention and its various embodiments can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Broadly, embodiments of the present invention provide a dental retractor having molded flexural support members to provide upper lip, lower lip, and cheek retraction. The retraction provides unobstructed access and visibility for facial/labial areas of the mouth during dental procedures. The lips are prevented from touching gingival tissue and tooth structure. Lips and soft tissue are retracted and held safely away from dental tools. Labial retraction is provided by flexible members which, when positioned in the use position, provide elevation and retraction for the labial surfaces both maxillary and mandibular, the counteractive forces of the maxillary and mandibular support members keep the lips retracted and away from treatment areas. As the flexural support members are flexed for labial retraction, the sides retract the cheeks due to the designed forces in the support member.

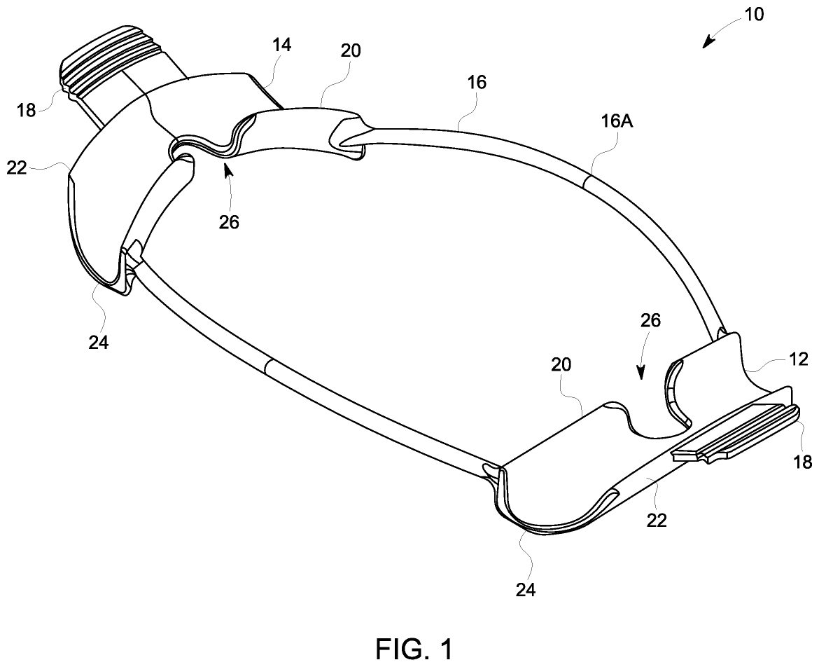

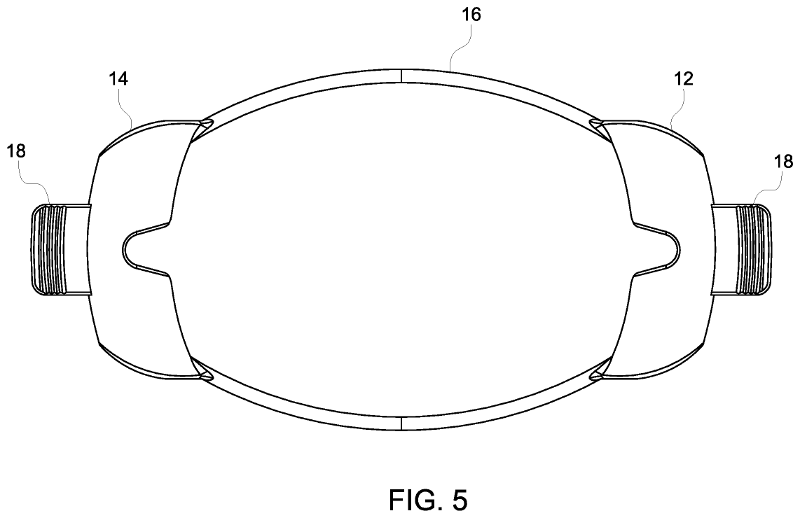

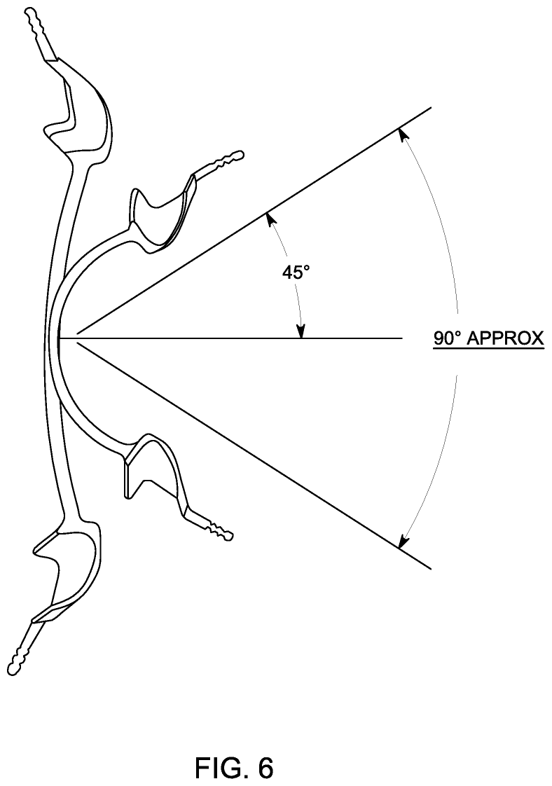

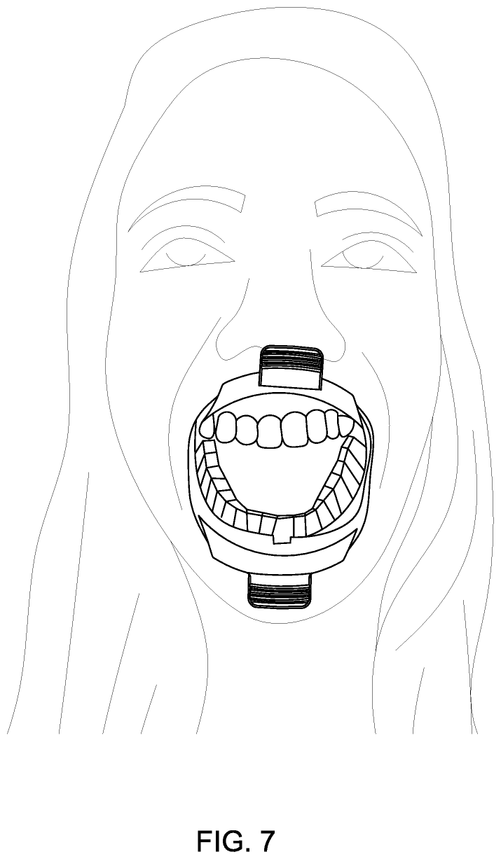

Referring to FIGS. 1 through 5, the dental retractor 10 (also referred to as retractor 10) can include upper and lower labial retraction members 12, 14 (also referred to an upper and lower lip saddles 12, 14) connected by a support element 16. One such support element 16 can connect left sides of the upper and lower labial retraction members 12, 14 and one support element 16 can connect right sides of the upper and lower labial retraction members 12, 14. The support element 16 may be arc shaped at rest and may be resiliently folded, as shown, for example, in FIG. 6. When the support element 16 is folded to an angle of about 90 degrees, the retractor 10 can be inserted into a patient, as shown in FIG. 7, where the resiliency of the support elements 16 provides an opening force to help maintain retraction, as discussed in greater detail below.

A medial portion 16A of the support element 16 can position itself against the buccal interior cheek tissue bilaterally. This causes a forward thrust in the opposing direction, away from the teeth. This thrust also pushes the lips forward and outward, away from the teeth and keeping the device away from sensitive gingival areas.

The first and second support members 16 can have a cross-sectional width in at least one direction of less than 10 mm, typically less than 5 mm, often less than 3 mm. For example, if the support members 16 are circular, the diameter would be less than about 10 mm. If the support members 16 have a rectangular cross section, the smaller width of this cross section would be no more than 10 mm. In other words, the support members 16 may be small, resiliently bendable members. The support members 16 may be made of the same material of the rest of the retractor or may include an interior portion being resiliently bendable, such as a metallic interior portion coated with a soft outer material.

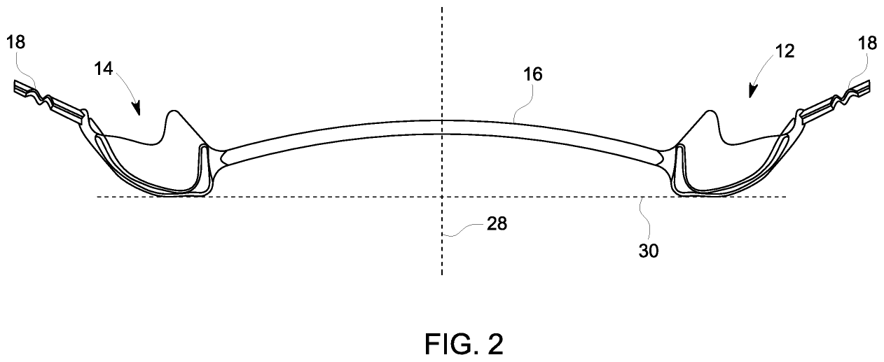

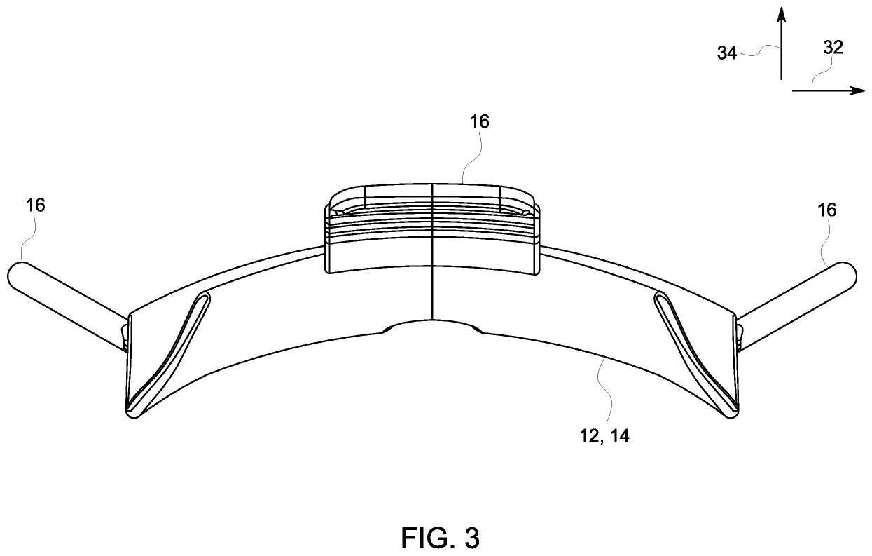

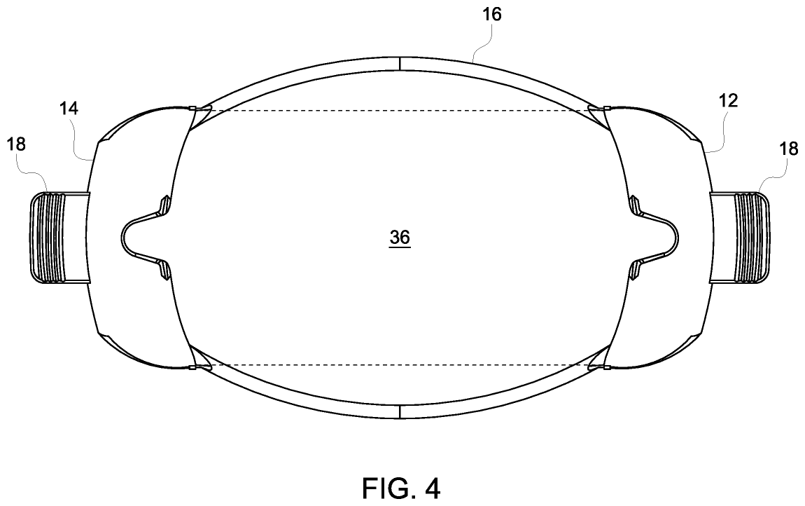

The upper lip saddle 12 and the lower lip saddle 14 can be disposed along a longitudinal axis 30 when the dental retractor 10 is at rest and not acted upon by any force, as shown in FIG. 2. The first and second support members 16 can provide resilient bending of the upper lip saddle 12 toward the lower lip saddle 14 and the first and second support members 16 are resiliently bent outward (as shown by arrow 32 in FIG. 3) from a space 36 (see FIG. 4) between the upper lip saddle 12 and the lower lip saddle 14 as the dental retractor 10 is bent to a use position (the right-hand image in FIG. 6).

The labial retraction members 12, 14 can be formed in a generally C-space, with the upper and lower labial retraction members 12, 14 open to the same direction. The retraction members 12, 14 can include an interior portion 20 that can be positioned where the gum tissue meets the lip tissue. Exterior portions 22 can be positioned, as shown in FIG. 7, on the exterior of the patient's lips. A sloped central portion 24 can provide a smooth, continuous arc connection the interior portion 20 with the exterior portion 22.

Tabs 18 can extend from the distal ends of the exterior portions 22. The tabs 18 may provide means for a provider to squeeze the upper and lower labial retraction members 12, 14 together (as shown in FIG. 6) to permit insertion into the patient. The tabs 18 may have a roughened outer surface to prevent finger slip during use. The tabs 18 allow clinicians to maneuver the mouthpiece for placement and to adjust the device laterally or vertically to enhance oral access and visibility.

The forward outward thrust of the retractor 10 manifests itself only when the device is positioned within the oral cavity in a bent or loaded (sprung) orientation, as shown in FIG. 7. This loaded position is approximately 90 degrees in deformation or bent from its molded or relaxed state.

Flexural resistance can assist in the retraction for multiple mouth morphologies where flexural support members 16 of the retractor 10 open after bending for insertion and self-seats device in the maxillary and mandibular vestibules. As can be seen, the support members 16 extend upward and outward in an arc shape when the device is at rest (as shown in FIGS. 1 through 5). When the provider squeezes the tabs 18 together (for example, toward a 90 degree angle as shown in FIG. 6), the support members 16 can not only provide the resistance to help with lip retraction, but also can resiliently extend outward, providing cheek retraction at the same time.

The retractor 10 can have a small cross section of lateral flexural support members 16 for ease of insertion and unobtrusiveness during dental procedures. Further, cut out reliefs 26 on lip saddles (the upper and lower labial retraction members 12, 14) for superior and inferior labial frenulum help ensure patient comfort.

In some embodiments, the retractor can be formed bi-laterally symmetrical. In other words, each side may be symmetrical about axis 28, as shown in FIG. 2. This allows for insertion without regards to which end is up or down.

The entire device can be made of a soft material, similar to gingival tissue, to allow for comfort against the gums. For example, the device can be made from soft flexible materials, such as plastics, rubber, foams or the like. The device can include molded bumpers to allow the lip retractors to enhance patient comfort by inhibiting impingement directly against gingival tissue. In some embodiments, the bumpers may be formed from a sponge-like material to permit absorption of excess irrigation fluids from the oral cavity.

As described above, the device can be molded so that the device is substantially flat or semi-flat in a storage, non-use condition to allow for simple mold designs. The device can be resiliently confirmed into a C-shape bent configuration during oral insertion and during use thereof.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Claim elements and steps herein may have been numbered and/or lettered solely as an aid in readability and understanding. Any such numbering and lettering in itself is not intended to and should not be taken to indicate the ordering of elements and/or steps in the claims.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. A dental retractor comprising:
an upper lip saddle;
a lower lip saddle, each of the upper lip saddle and the lower lip saddle having an interior and an exterior, the interior being positioned between a user's lip and gum when the dental retractor is placed on a user, the exterior being positioned on an outer portion of the user's lips with the dental retractor is placed on the user;
a first support member joining a left side of the interior of the lower lip saddle with a left side of the interior of the upper lip saddle; and
a second support member joining a right side of the interior of the lower lip saddle with a right side of the interior of the upper lip saddle, wherein
the upper lip saddle and the lower lip saddle being disposed along a longitudinal axis when the dental retractor is at rest and not acted upon by any force;
the first and second support members provide resilient bending of the upper lip saddle toward the lower lip saddle; and
the first and second support members resiliently bend outward from a space between the upper lip saddle and the lower lip saddle when the dental retractor is bent to a use position.

2. The dental retractor of claim 1, further comprising cut out reliefs formed on a central portion of the interior of the upper lip saddle and the lower lip saddle.

3. The dental retractor of claim 1, wherein each of the upper lip saddle and the lower lip saddle is formed in C-shape.

4. The dental retractor of claim 1, further comprising a tab on a central portion of the exterior of the upper lip saddle and the lower lip saddle.

5. The dental retractor of claim 1, wherein the first and second support members have a cross-sectional width in at least one direction of less than 5 mm.

6. The dental retractor of claim 1, wherein the first and second support members have a round cross section with a diameter less than 5 mm.

7. The dental retractor of claim 1, wherein an upper half of the dental retractor, having the upper lip saddle, is symmetrical with a lower half of the dental retractor, having the lower lip saddle.

8. The dental retractor of claim 1, wherein the first and second support members press against buccal interior cheek tissue bilaterally to provide retraction when the dental retractor is positioned in a user in a use position.

9. A dental retractor comprising:
an upper lip saddle;
a lower lip saddle, each of the upper lip saddle and the lower lip saddle having an interior and an exterior, the interior being positioned between a user's lip and gum when the dental retractor is placed on a user, the exterior being positioned on an outer portion of the user's lips with the dental retractor is placed on the user;
a tab on a central portion of each of the exterior of the upper lip saddle and the lower lip saddle;
a first support member joining a left side of the interior of the lower lip saddle with a left side of the interior of the upper lip saddle; and
a second support member joining a right side of the interior of the lower lip saddle with a right side of the interior of the upper lip saddle, wherein
the upper lip saddle and the lower lip saddle being disposed along a longitudinal axis when the dental retractor is at rest and not acted upon by any force;
the first and second support members provide resilient bending of the upper lip saddle toward the lower lip saddle;
the first and second support members resiliently bend outward from a space between the upper lip saddle and the lower lip saddle when the dental retractor is bent to a use position; and
an upper half of the dental retractor, having the upper lip saddle, is symmetrical with a lower half of the dental retractor, having the lower lip saddle.

10. The dental retractor of claim 9, further comprising cut out reliefs formed on a central portion of the interior of the upper lip saddle and the lower lip saddle.

11. The dental retractor of claim 9, wherein each of the upper lip saddle and the lower lip saddle is formed in C-shape.

12. The dental retractor of claim 9, wherein the first and second support members have a cross-sectional width in at least one direction of less than 10 mm.

13. The dental retractor of claim 9, wherein the first and second support members have a round cross section with a diameter less than 10 mm.

14. The dental retractor of claim 9, wherein the first and second support members press against buccal interior cheek tissue bilaterally to provide retraction when the dental retractor is positioned in a user in a use position.

15. A method of retracting a user's lips and cheeks comprising:
squeezing the tabs of the dental retractor of claim 9 toward each other to resiliently bend the dental retractor into an angle of about 90 degrees; and
inserting the dental retractor into a user's mouth, where the upper lip saddle is positioned about a user's upper lip, the lower lip saddle is positioned about a user's lower lip, and each of the first and second support members press against an interior of respective left and right cheeks of a user.

16. The method of claim 15, further comprising positioning cut out reliefs formed on a central portion of the interior of the upper lip saddle and the lower lip saddle to encircle superior and inferior labial frenulum of the user.

17. The method of claim 15, wherein each of the upper lip saddle and the lower lip saddle is formed in C-shape.

18. The method of claim 15, wherein the first and second support members have a cross-sectional width in at least one direction of less than 10 mm.

19. The method of claim 15, wherein the first and second support members have a round cross section with a diameter less than 10 mm.

20. The method of claim 15, further comprising providing a thrust from resiliently moving the tabs toward each other, the thrust pushing the upper and lower lips forward and outward, away from teeth and keeping the dental retractor away from sensitive gingival areas.
